(19) United States
(12) Patent Application Publication
(10) Pub. No.: US 2025/0041356 A1
(43) Pub. Date: Feb. 6, 2025

(54) COMBINATION CANCER THERAPY

(71) Applicant: Prokarium Limited, London (GB)

(72) Inventors: Matthieu Besneux, London (GB); Nicholas Glanville, London (GB); Livija Deban, London (GB)

(21) Appl. No.: 18/716,430

(22) PCT Filed: Dec. 9, 2022
(86) PCT No.: PCT/EP2022/085258

§ 371 Date: Jun. 4, 2024

(30) Foreign Application Priority Data
Dec. 9, 2021 (EP) 21213559.4

(51) Int. Cl. A61K 35/74; A61K 31/675; A61K 31/704; A61K 31/7068; A61K 45/06; A61P 35/04

(52) U.S. Cl. CPC A61K 35/74; A61K 31/675; A61K 31/704; A61K 31/7068; A61K 45/06; A61P 35/04

(57) ABSTRACT

The present invention relates to the prevention and/or treatment of cancer in a subject, wherein the subject is administered a Gram-negative bacterium in a first treatment phase and a chemotherapy agent in a second treatment phase.

### FIELD OF THE INVENTION

The present invention relates to the field of cancer therapy. In particular, the present invention relates to a method of preventing, treating or inhibiting the development of neoplastic disease in a subject.

### BACKGROUND OF THE INVENTION

The field of cancer therapy is continually evolving with new therapies as our understanding of the underlying mechanisms associated with cancer formation and progression improve. However, despite the significant progress made in the field of cancer, chemotherapy remains the mainstay of treatment of cancer.

Chemotherapy is a term used to encompass drug treatments that use cytotoxic chemicals to kill fast-growing cells in the body. As such, these drugs are most often used to treat cancer cells due to their associated enhanced growth and multiplication rate compared to most other cells in the body. However, chemotherapy can be associated with serious adverse effects and other numerous shortcomings, such as inadequate drug concentrations in tumours, occurrence of systemic toxicity and induction of drug resistance. Accordingly, numerous methods have been developed whereby the efficacy of chemotherapy can either be maintained or improved whilst adequately controlling the resulting side effects.

For example, one such method is the use of chemotherapy drugs in combination with bacteria. It has been shown that such a combination can significantly improve the effects of chemotherapy treatments in murine models (Jia et al., 2007, int. J. Cancer: 121, 666-674). However, the bacteria in this study were administered directly to the tumour, and thus required to be in close proximity to the tumour in order to have an effect. It was additionally found that in order to overcome lethal toxicity of the combination of bacteria and chemotherapy, it was necessary to optimize the dosing regimen such that the bacteria was administered 12 days post chemotherapy treatment. In other methods, it has been shown that bacteria, specifically Salmonella typhimurium, can be co-administered with a chemotherapy agent such that a synergistic effect is achieved (see WO2018106754).

However, despite the move to using chemotherapy in combination with additional treatments, there is still room for improvement in the level of efficacy that is currently observed. Accordingly, there remains a significant need in the cancer field for methods which result in enhanced efficacy of chemotherapies.

### SUMMARY OF THE INVENTION

The present invention provides an effective method for treating and/or preventing neoplastic disease, including metastasis, in a subject by administering live attenuated Gram-negative bacteria in combination with a chemotherapy. Such a method allows for the efficacy of current chemotherapy regimens to be improved, as well as the level of normal tissue damage during/after the chemotherapy to be reduced.

In a first aspect of the invention, there is a live attenuated Gram-negative bacterium for use in the prevention or treatment of a neoplastic disease in a subject undergoing, or intended to undergo, chemotherapy with a chemotherapy agent, wherein the live attenuated Gram-negative bacterium is for administration in a first treatment phase and the chemotherapy agent is for administration in a second treatment phase.

In a second aspect of the invention, there is a method of preventing or treating a neoplastic disease in a subject wherein the method comprises administering to the subject, (i) a live attenuated Gram-negative bacterium in a first treatment phase and (ii) a chemotherapy agent in a second treatment phase, wherein said method results in an enhanced therapeutic efficacy relative to the administration of the bacterium or chemotherapy agent alone.

### DESCRIPTION OF FIGURES

FIG. 1 shows a schematic of the mechanism of action of the conditioning effect of Salmonella on chemotherapy.

FIG. 2 shows orally administered Salmonella induces significant changes in the long-term phenotype of systemic myeloid cells. A&B) Graphs show median fluorescence intensity of markers CD80, CD86 and PD-L1 on viable, CD11c^{high}, HLA-DR⁺, CD11b^{+/-}, PDCA-1⁻ conventional dendritic cells and viable, CD11c^{-/low}, PDCA1⁺, HLA-DR^{-/Int}, CD11b⁻ plasmacytoid dendritic cells; C&D) Graphs show median fluorescence intensity of markers PD-L1, CD80 and HLA-DR on viable, CD11c⁻, CD11b⁺, Ly6C⁺, F4/80⁻ monocytes and viable, CD11c⁻, CD11b⁺, Ly6C⁻, F4/80⁺ macrophages. n=4 or 5 mice/group.

FIG. 3 shows a time-course of Salmonella-induced phenotypic changes. A) Experiment schematic detailing the timeline of experiments; B) Graphs show median fluorescence intensity of markers CD80 and CD86 as a percentage of the PBS control group mean for viable, CD11c^{high}, HLA-DR⁺, CD11b^{+/-}, PDCA-1⁻ conventional dendritic cells (CDC), and viable, CD11c^{-/low}, PDCA1⁺, HLA-DR^{-/Int} CD11b⁻ plasmacytoid dendritic cells (pDC). Shown are means of n=4-5 mice/group; C) Graphs show median fluorescence intensity of markers CD80, PD-L1 and HLA-DR as a percentage the PBS control group mean for; viable, CD11c⁻, CD11b⁺, Ly6C⁺, F4/80⁻ monocytes, and CD11c⁻, CD11b⁺, Ly6C⁻, F4/80⁺ macrophages. Shown are means of n=4-5 mice/group.

FIG. 4 shows oral administration of Salmonella results in enhanced myelopoiesis. A) Graphs show the % of lineage negative (CD5⁻, CD11b⁻, B220⁻, GR-1⁻, Terr-119⁻, Ly-6B.2⁻) viable cells expressing both c-Kit and Sca-1, termed LKS cells of total bone marrow cells. n=4-5 mice/group; B) Representative flow cytometry plots showing increase in viable LKS cells in the bone marrow of animals treated orally with Salmonella; C) % viable LKS cells of total bone marrow cells at day 14 after Salmonella treatment was correlated with % spleen monocytes (viable, CD11c−, CD11b+, Ly6C+, F4/80− cells) at the same timepoint using Spearman rank correlation.

FIG. 5 shows orally administered Salmonella induces a hyperresponsive state in systemic dendritic cells that lasts at least 14 days. n=5 mice per group from a single study; bars are mean+/−SEM; Statistics indicated are Mann-Whitney test.

FIG. 6 shows in vitro data demonstrating the hyperresponsiveness of primary human monocytes in response to Pathogen Associated Molecular Patterns (PAMPs) and Danger Associated Molecular Patterns (DAMPS), wherein the cells have previously been conditioned with media only, Salmonella Typhi or β-glucan (a fungal/bacterial moiety).

FIG. 7A shows schematic demonstration of the timeline of oral systemic administration of Salmonella Typhimurium, followed by systemic administration of cyclophosphamide in a syngeneic orthotopic 4T1 murine breast cancer model.

FIG. 7B shows primary tumour volume (mm3) of 4T1 tumour-bearing mice treated with Salmonella Typhimurium MD58, cyclophosphamide, or both (n=15 mice per group). Female BALB/c mice were pre-treated orally with Salmonella MD58 or PBS control. On day 0 mice were inoculated in the mammary fat pad with 4T1-Luc2-1A4 tumour cells. Primary tumour volumes were measured 3 times per week for 52 days. Statistical comparisons are MD58+cyclophosphamide vs. PBS+cyclophosphamide using mix effects model and Sidaks multiple comparisons test (degree of significance not indicated).

FIG. 7C shows spontaneous lung metastasis in 4T1 tumour-bearing mice treated with Salmonella Typhimurium MD58, cyclophosphamide, or both (n=10 mice per group). Female BALB/c mice were treated as in FIG. 7A&B. Lung tumour burden was measured by bioluminescent imaging (BLI) at day 31 post tumour inoculation. Statistical comparisons are individual Mann-Whitney tests for selected comparisons only.

FIG. 7D shows percentage survival of 4T1 tumour-bearing mice treated with Salmonella Typhimurium MD58, cyclophosphamide, or both (n=15 mice per group). Female BALB/c mice were treated as in FIG. 7A-C. Survival was monitored over 52 days. Both cyclophosphamide treated groups had significantly better survival than PBS+saline control but there was no statistical difference between PBS+cyclophosphamide and MD58+cyclophosphamide groups. Statistics are log-rank (Mantel-Cox) test.

FIG. 8A shows schematic demonstration of the timeline of oral systemic administration of Salmonella Typhimurium, followed by intravenous administration of 4T1 murine breast cancer cell, and then followed by systemic cyclophosphamide.

FIG. 8B shows experimental lung metastasis in 4T1 tumour-bearing mice treated with Salmonella Typhimurium MD58, cyclophosphamide, or both (n=10 mice per group). Female BALB/c mice were treated as in FIG. 8A. Lung tumour burden was measured by bioluminescent imaging (BLI) at day 5 and 12 post tumour inoculation. Each bar represents group medians. Statistical comparisons are individual Mann-Whitney tests for selected comparisons only.

FIG. 8C shows Percentage survival of mice bearing experimental metastasis of 4T1 breast cancer cells treated with Salmonella Typhimurium MD58, cyclophosphamide, or both (n=15 mice per group). Female BALB/c mice were treated as in FIG. 8A&B. Survival was monitored over 25 days. Statistical comparisons shown are indicated groups vs. PBS+cyclophosphamide only by log-rank (Mantel-Cox) test.

FIG. 9A shows schematic demonstration of the timeline of oral systemic administration of Salmonella Typhimurium, followed by intravenous administration of 4T1 murine breast cancer cell, and then followed by intraperitoneal gemcitabine.

FIG. 9B shows experimental lung metastasis in 4T1 tumour-bearing mice treated with Salmonella Typhimurium MD58, gemcitabine, or both (n=10 mice per group). Female BALB/c mice were treated as in FIG. 9A. Lung tumour burden was measured by bioluminescent imaging (BLI) at day 6 post tumour inoculation. Each bar represents group medians. Statistical comparisons are individual Mann-Whitney tests for selected comparisons only.

FIG. 10A shows schematic demonstration of the timeline of oral systemic administration of Salmonella Typhimurium, followed by intravenous administration of LL/2-Luc-M38 murine lung cancer cell, and then followed by systemic cyclophosphamide.

FIG. 10B shows Lung tumour burden in LL/2-luc tumour-bearing mice treated with Salmonella Typhimurium MD58, cyclophosphamide, or both (n=10 mice per group). Female albino C57BL/6 mice were treated as in FIG. 10A. Lung tumour burden was measured by bioluminescent imaging (BLI) at day 10 post tumour inoculation. Each bar represents group medians. Statistical comparison is individual Mann-Whitney test for selected comparison only (n.s.).

FIG. 11A shows schematic demonstration of the timeline of oral systemic administration of Salmonella Typhimurium, followed by intravenous administration of 4T1 murine breast cancer cells, and then followed by systemic administration of doxorubicin.

FIG. 11B shows experimental lung metastasis in 4T1 tumour-bearing mice treated with Salmonella Typhimurium MD58, doxorubicin, or both (n=10 mice per group). Female BALB/c mice were treated as in FIG. 11A. Lung tumour burden was measured by bioluminescent imaging (BLI) at day 6 post tumour inoculation. Each bar represents group medians. Statistical comparisons are individual Mann-Whitney tests for selected comparisons only.

### DETAILED DESCRIPTION

In order that the present invention may be more readily understood, certain terms are first defined. Additional definitions are set forth throughout the detailed description.

As used herein, the term “attenuated” in the context of the present invention, refers to the alteration of a microorganism to reduce its pathogenicity, rendering it harmless to the host, whilst maintaining its viability. This method is commonly used in the development of vaccines due to its ability to elicit a highly specific immune response whilst maintaining an acceptable safety profile. Development of such attenuated microorganisms may involve a number of methods, examples include, but are not limited to, passing the pathogens under in vitro conditions until virulence is lost, chemical mutagenesis and genetic engineering techniques, for example, by inactivating mutations or attenuating mutations. The terms “inactivating mutations” and “attenuating mutations” are used interchangeably and refer to modifications of the natural genetic code of a particular gene or gene promoter associated with that gene, such as modification by changing the nucleotide code or deleting sections of nucleotide or adding non-coding nucleotides or non-natural nucleotides, such that the particular gene is either not transcribed or translated appropriately or is expressed into a non-active protein such that the gene's natural function is abolished or reduced to such an extent that it is not measurable. Thus, the mutation of the gene inactivates that gene's function or the function of the protein which that gene encodes.

By “non-natural bacterium or bacteria” we mean a bacterial (prokaryotic) cell that has been genetically modified or “engineered” such that it is altered with respect to the naturally occurring cell. Such genetic modification may for example be the incorporation of additional genetic information into the cell, modification of existing genetic information or indeed deletion of existing genetic information. This may be achieved, for example, by way of transfection of a recombinant plasmid into the cell or modifications on directly to the bacterial genome.

As used herein, the term “chemotherapy”, “chemotherapeutic” and “chemotherapy agent” are used interchangeably and refer to the anti-cancer drugs used in the prevention and/or treatment of cancer. Specifically, it refers to anti-cancer chemicals that prevent cancer cells from growing and dividing. Chemotherapy agents include, but are not limited to, alkylating agents, plant alkaloids, antitumour antibiotics, antimetabolites and/or topoisomerase inhibitors, or any combination thereof. Preferably, the chemotherapy agent may be selected from the group comprising cisplatin, gemcitabine, carboplatin, methotrexate, vinblastine, doxorubicin, or any combination thereof.

As used herein, the terms “first treatment phase” and “second treatment phase” refer to a course of treatment whereby the first treatment phase and the second treatment phase are temporally spaced, such that there is a gap between the first and second treatment phase where the patient/subject is not receiving the Gram-negative bacteria herein disclosed, or the chemotherapy herein disclosed. In other words, the Gram-negative bacteria is given to the subject to be treated before the chemotherapy. In a preferred embodiment, the first treatment phase is initiated at least one week prior to the second treatment phase. The first treatment phase may be initiated at least two weeks prior, at least three weeks prior, at least four weeks prior to the second treatment phase. Even more preferably, the first treatment phase is initiated at least ten days prior to the second treatment phase. The first treatment phase may be administered to allow sufficient time to generate a systemic immune response in the subject prior to the second treatment phase.

As used herein, the term “heterologous polynucleotide” refers to a polynucleotide that has been introduced into the Gram-negative bacteria i.e., the introduction of a polynucleotide that was not previously present. The polynucleotide may be exogenous to the bacterium, whereby these terms have their normal meaning in the art. For an endogenous polynucleotide, this may comprise introducing an additional copy or copies of said one or more endogenous polynucleotide in a heterologous manner. The endogenous polynucleotide or polynucleotides may also comprise introducing dominant variants of said polynucleotide or polynucleotides into the host bacterium, whereby “dominant” refers to the ability of the heterologous polynucleotide to functionally out-compete the naturally occurring endogenous counterpart. The heterologous polynucleotide in the context of the present invention may encode for a target polypeptide intended for delivery i.e., for export and secretion, in a subject. The resulting polypeptide is also referred to herein as “cargo” or a “cargo molecule”. Accordingly, the Gram-negative bacteria herein disclosed may, in addition to its effects herein disclosed, act as a “delivery vehicle” or “carrier” for the chosen cargo if so desired. The skilled person will readily understand that the cargo to be delivered will be dependent on a number of factors, including the type and severity of cancer to be treated. Preferably, the heterologous polynucleotide encodes a therapeutic protein, for example, a cytokine and/or chemokine. More preferably, the heterologous polynucleotide encodes IL-15, IL-21, IFNα, CXCL9, CXCL10, IL-18, IL-27 or any combinations thereof.

The terms “tumour,” “cancer” and “neoplasia” are used interchangeably and refer to a cell or population of cells whose growth, proliferation or survival is greater than growth, proliferation or survival of a normal counterpart cell, e.g. a cell proliferative or differentiative disorder. Typically, the growth is uncontrolled. The term “malignancy” refers to invasion of nearby tissue. The term “metastasis” refers to spread or dissemination of a tumour, cancer or neoplasia to other sites, locations or regions within the subject, in which the sites, locations or regions are distinct from the primary tumour or cancer.

The terms “effective amount” or “pharmaceutically effective amount” refer to a sufficient amount of an agent to provide the desired biological or therapeutic result. That result can be reduction, amelioration, palliation, lessening, delaying, and/or alleviation of one or more of the signs, symptoms, or causes of a disease, or any other desired alteration of a biological system. In reference to cancer, an effective amount may comprise an amount sufficient to cause a tumour to shrink and/or to decrease the growth rate of the tumour (such as to suppress tumour growth) or to prevent or delay other unwanted cell proliferation. In some embodiments, an effective amount is an amount sufficient to delay development, or prolong survival or induce stabilisation of the cancer or tumour.

In some embodiments, a therapeutically effective amount is an amount sufficient to prevent or delay recurrence. A therapeutically effective amount can be administered in one or more administrations. The therapeutically effective amount of the drug or combination may result in one or more of the following: (i) reduce the number of cancer cells; (ii) reduce tumour size; (iii) inhibit, retard, slow to some extent and preferably stop cancer cell infiltration into peripheral organs; (iv) inhibit (i.e., slow to some extent and preferably stop) tumour metastasis; (v) inhibit tumour growth; (vi) prevent or delay occurrence and/or recurrence of tumour; and/or (vii) relieve to some extent one or more of the symptoms associated with the cancer.

For example, for the treatment of tumours, a “therapeutically effective dosage” may induce tumour shrinkage by at least about 5% relative to baseline measurement, such as at least about 10%, or about 20%, or about 60% or more. The baseline measurement may be derived from untreated subjects.

A therapeutically effective amount of a therapeutic compound can decrease tumour size, or otherwise ameliorate symptoms in a subject. One of ordinary skill in the art would be able to determine such amounts based on such factors as the subject's size, the severity of the subject's symptoms, and the particular composition or route of administration selected.

The term “immune response” in the context of the present invention refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells. Preferably, the immune response generated by the Gram-negative bacteria herein disclosed is systemic. As used herein, the terms “systemic” and “systemically activated” are used interchangeably and in the context of the present invention refers to a widespread immune response throughout the body of a subject, as opposed to a local, spatially-restricted response. Preferably, the systemic immune response involves the activation and/or maturation of myeloid cells, for example, dendritic cells, monocytes and/or macrophages and in the context of the present invention is thought to help “condition” the immune system of the subject, such that the subject is more responsive to a cancer therapy, for example, a chemotherapy. Accordingly, the Gram-negative bacteria may act to “prime”, “boost”, “amplify”, “enhance”, “improve”, “augment”, “pre-activate” or “promote” the immune response of a subject prior to administering the chemotherapy. The aforementioned terms are used interchangeably with the term “conditioned”.

The term “treatment” or “therapy” refers to administering an active agent with the purpose to cure, heal, alleviate, relieve, alter, remedy, ameliorate, improve, or affect a condition (e.g., a disease), the symptoms of the condition, or to prevent or delay the onset of the symptoms, complications, biochemical indicia of a disease, or otherwise arrest or inhibit further development of the disease, condition, or disorder in a statistically significant manner.

As used herein, the term “subject” is intended to include human and non-human animals. Preferred subjects include human patients in need of enhanced efficacy of any given chemotherapy. The methods are particularly suitable for treating human patients having a disorder that can be treated by augmenting the immune response. In a particular embodiment, the methods are particularly suitable for treatment of cancer in vivo.

The use of the alternative (e.g., “or”) should be understood to mean either one, both, or any combination thereof of the alternatives. As used herein, the indefinite articles “a” or “an” should be understood to refer to “one or more” of any recited or enumerated component.

As used herein, “about” means within an acceptable error range for the particular value as determined by one of ordinary skill in the art, which will depend in part on how the value is measured or determined, i.e., the limitations of the measurement system. For example, “about” can mean within 1 or more than 1 standard deviation per the practice in the art. Alternatively, “about” can mean a range of up to 20%.

When particular values are provided in the application and claims, unless otherwise stated, the meaning of “about” should be assumed to be within an acceptable error range for that particular value.

As used herein, the terms “recombinant” and “recombinant strain(s)” are used interchangeably and in the context of the present invention refers to a strain of Gram-negative bacteria that has undergone genetic engineering such that the bacterial DNA has been altered by the introduction of new DNA. Recombinant DNA methods commonly involve the introduction of new DNA via a vector, for example, a plasmid. Such methods are well known to those skilled in the art. Use of recombinant strains of bacteria may confer advantageous properties to the bacterial strain, such as prolonged activity, eliciting a stronger immune response in a subject, or introduction of a desired molecule.

As used herein, the terms “non-recombinant” and “non-recombinant strain(s)” are used interchangeably and in the context of the present invention refers to the fact that these strains do not contain genes or gene fragments from eukaryotic organisms. As such, the non-recombinant strains herein disclosed do not act as “carrier strains” for the purpose of delivering therapeutic molecules to a subject/patient. Accordingly, the non-recombinant strains herein disclosed do not encode eukaryotic heterologous DNA, or eukaryotic heterologous DNA that encodes a therapeutic molecule, or eukaryotic DNA that encodes for proteins, or fragments thereof, that are intended as antigens.

Therefore, in any one embodiment of the invention, the Gram-negative bacteria may be non-recombinant and does not act as a “carrier” strain for the purpose of the delivery of a therapeutic molecules, or delivery of DNA molecule that encodes for a therapeutic molecule.

The present invention provides an effective and safe method in which the efficacy of a chemotherapeutic agent can be enhanced, thus improving cancer outcomes in subjects at various doses of chemotherapy. As such, the method herein disclosed may also provide a method in which the side-effects of chemotherapies are minimised due to their improved efficacy, thus reducing the length of time that the subject may need to undergo cancer treatment.

Accordingly, in a first aspect, there is a live attenuated Gram-negative bacterium for use in the prevention or treatment of a neoplastic disease in a subject undergoing, or intended to undergo, chemotherapy with a chemotherapy agent, wherein the live attenuated Gram-negative bacterium is for administration in a first treatment phase and the chemotherapy agent is for administration in a second treatment phase.

Chemotherapy drugs are, by design, cytotoxic to enable the destruction of cancerous cells. During the process by which cancerous cells are destroyed, tissue damage associated compounds (DAMPs) are released in a process call immunogenic cell death. Examples of DAMPs include heat shock proteins and HMGB1. Immunogenic cell death involves changes in the composition of the cell surface, as well as the release of soluble mediators, occurring in a defined temporal sequence. Such signals are known to aid in the activation of the immune system against cancer.

The inventors of the present invention have surprisingly found that administration of Gram-negative bacteria prior to the administration of chemotherapy results in significant changes in myeloid cells of the immune system of the subject, thus inducing high levels of myeloid cell responsiveness to tissue damage-associated compounds released following chemotherapy. As a result, an enhanced anti-tumour effect is produced. Accordingly, the live attenuated Gram-negative bacteria are considered as a “conditioning” agent of the subject's immune system, resulting in a systemic immune response and the subsequent ability of the subject's immune system to be able to mount a more effective innate immune response as well as an effective adaptive response to a neoplastic disease following chemotherapy treatment.

The live attenuated Gram-negative bacteria are to be administered to elicit a systemic immune response, and therefore the preferred routes of administration are oral, intravenous, subcutaneous, intradermal and intramuscular.

The live attenuated Gram-negative bacteria may preferably be formulated for oral delivery. Formulations suitable for these routes of delivery will be apparent to the skilled person. Preferably, the live attenuated Gram-negative bacteria is a liquid frozen formulation or lyophilised by a process such as freeze-drying and stored appropriately. Alternatively, the live attenuated Gram-negative bacteria may be dispensed into enterically coated capsules. Where an encapsulated formulation is used, the lyophilised bacteria may be mixed with a bile-adsorbing resin, such as cholestyramine, to enhance survival when released from the capsule into the small intestine (for further details, see WO 2010/079343). The particular formulation of the bacteria may vary depending on a variety of factors, for example, the target patient population i.e., young children, adolescents or adults. The live attenuated Gram-negative bacteria may be formulated in a composition comprising any other suitable adjuvant, diluent or excipient. Suitable adjuvants, diluents or excipients include, but are not limited to, disodium hydrogen phosphate, soya peptone, potassium dihydrogen phosphate, ammonium chloride, sodium chloride, magnesium sulphate, calcium chloride, sucrose, sterile saline and sterile water.

Oral administration of the live attenuated Gram-negative bacteria has numerous advantages over other routes of administration. Firstly, oral administration is a non-invasive method of administration, an important factor in improving and maintaining patient compliance. This is particularly true in cancer subjects who are regularly undergoing invasive and, most likely, unpleasant procedures. Secondly, oral administration of the live attenuated Gram-negative bacteria herein disclosed provides the advantageous effect of improved efficacy of chemotherapy without the need for intratumoural delivery of the live attenuated Gram-negative bacteria. Accordingly, the utility of the present invention is not limited by the location of the tumour to be treated, for example, if a tumour is in a hard-to-reach place.

It is envisaged that any live attenuated Gram-negative bacteria capable of producing the required immune response in a subject may be used in the present invention. Examples of Gram-negative bacteria for use in the present invention include, but are not limited to, Escherichia coli, Salmonella, Shigella, Pseudomonas, Moraxella, Helicobacter, Stenotrophomonas, Bdellovibrio, Legionella, Chlamydia and Yersinia, or any combination thereof. Gram-negative bacteria can be readily identified and differentiated from Gram-positive bacteria via Gram's differential staining technique, where Gram-negative bacteria do not retain the crystal violet stain.

Preferably, the live attenuated Gram-negative bacteria of the invention may be a Salmonella spp. Examples of Salmonella species for use in the present invention are Salmonella enterica and Salmonella bongori. Salmonella enterica can be further sub-divided into different serotypes or serovars. Examples of said serotypes or serovars for use in the present invention are Salmonella enterica Typhi, Salmonella enterica Paratyphi A, Salmonella enterica Paratyphi B, Salmonella enterica Paratyphi C, Salmonella enterica Typhimurium and Salmonella enterica Enteritidis, or any combination thereof. In a preferred embodiment, the live attenuated Gram-negative bacterium may be Salmonella enterica serovar Typhi and/or Salmonella enterica Typhimurium. In a most preferred embodiment, the live attenuated Gram-negative bacterium is Salmonella enterica serovar Typhi.

The live attenuated Gram-negative bacteria of the invention may comprise a genetically modified non-natural bacterium. As would be understood by a person of skill in the art, genes may be mutated by a number of well-known methods in the art, such as homologous recombination with recombinant plasmids targeted to the gene of interest, in which case an engineered gene with homology to the target gene is incorporated into an appropriate nucleic acid vector (such as a plasmid or a bacteriophage), which is transfected into the target cell. The homologous engineered gene is then recombined with the natural gene to either replace or mutate it to achieve the desired inactivated mutation. Such modification may be in the coding part of the gene or any regulatory portions, such as the promoter region. As would be understood by a person of skill in the art, any appropriate genetic modification technique may be used to mutate the genes of interest, such as the CRISPR/Cas system, e.g. CRISPR/Cas 9.

Thus, numerous methods and techniques for genetically engineering bacterial strains will be well known to the person skilled in the art. These techniques include those required for introducing heterologous genes into the bacteria either via chromosomal integration or via the introduction of a stable autosomal self-replicating genetic element. Exemplary methods for genetically modifying (also referred to as “transforming” or “engineering”) bacterial cells include bacteriophage infection, transduction, conjugation, lipofection or electroporation. A general discussion on these and other methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); which are hereby incorporated by reference.

Accordingly, the live attenuated Gram-negative bacteria may have had its genetic make-up altered in some form, for example, via genetic engineering, or via chemical mutagenesis, to induce a change, for example, a mutation, an addition or deletion. The live attenuated Gram-negative bacteria may be genetically modified such that the live attenuated Gram-negative bacteria is a recombinant strain of bacteria that may, or may not, further comprise a heterologous polynucleotide encoding a polypeptide. Said polypeptide may be a therapeutic molecule in of itself or a molecule for supporting/enhancing the effect of the Gram-negative bacteria. Alternatively, the live attenuated Gram-negative bacteria may be a non-recombinant strain of bacteria.

It is envisaged that any live attenuated Gram-negative bacteria capable of inducing the systemic immune response of a subject in accordance with the invention may be used. In a preferred embodiment, any attenuated, non-pathogenic, Salmonella enterica serovar Typhi or Typhimurium strain may be used. In a further preferred embodiment, the live attenuated Gram-negative bacterium may be selected from the group comprising Ty21a, CVD 908-htrA, CVD 909, Ty800, M01ZH09 (used interchangeably with “ZH9”), ZH9PA, x9633, x639, x9640, x8444, DTY88, MD58, WT05, ZH26, SL7838, SL7207, VNP20009, A1-R, or any combinations thereof. In a preferred embodiment, the live attenuated bacteria is M01ZH09 or MD58.

Accordingly, wherein the live attenuated Gram-negative bacteria is a genetically modified non-natural bacterium, it is preferred that said genetically modified non-natural bacteria is derived from Salmonella spp. It is further preferred that said Salmonella spp. may comprise an attenuating mutation in a Salmonella Pathogenicity Island 2 (SPI-2) gene and/or an attenuating mutation in a second gene. Preferably, the genetically modified non-natural bacteria is derived from Salmonella spp. and comprises both an attenuating mutation in a SPI-2 gene and an attenuating mutation in a second gene. Suitable genes and details of such a live attenuated Salmonella microorganism is as described in WO 2000/68261, which is hereby incorporated by reference in its entirety.

The SPI-2 gene may be an ssa gene. For example, the invention includes an attenuating mutation in one or more of ssaV, ssaJ, ssaU, ssak, ssaL, ssaM, ssaO, ssaP, ssaQ, ssaR, ssaS, ssaT, ssaD, ssaE, ssaG, ssal, ssaC and ssaH. Preferably, the attenuating mutation is in the ssaV or ssal gene. Even more preferably, the attenuating mutation is in the ssaV gene.

The genetically engineered Salmonella microorganism may also comprise an attenuating mutation in a second gene, which may or may not be in the SPI-2 region. The mutation may be outside of the SPI-2 region and involved in the biosynthesis of aromatic compound. For example, the invention may include an attenuating mutation in an aro gene. In a preferred embodiment, the aro gene is aroA or aroC. Even more preferably, the aro gene is aroC.

When the genetically engineered Salmonella microorganism comprises a double attenuating mutation, both mutations may be in the SPI-2 gene or both mutations may be in a second gene, which may or may not be in the SPI-2 region. Preferably, the genetically engineered Salmonella microorganism comprises an attenuating mutation in the ssaV gene and an aro gene, even more preferably, wherein the aro gene is aroC.

In yet another embodiment, the genetically engineered microorganism may be derived from a Salmonella microorganism and may comprise inactivating mutations in one or more genes selected from pltA, pltB, cdtB and ttsA and further comprises attenuating mutations in one or more genes selected from aroA and/or aroC and/or ssaV. Preferably, the attenuating mutations are in aroC and ssaV. Details of said genes and mutations are as described in WO 2019/110819, which is hereby incorporated by reference in its entirety.

The live attenuated Gram-negative bacteria herein disclosed may comprise a heterologous polynucleotide encoding a target protein or peptide. The heterologous polynucleotide may encode for a target protein or peptide that has anti-cancer properties in of itself, and/or the heterologous polynucleotide may encode for a protein or peptide that supports and/or enhances the properties of the live attenuated Gram-negative bacteria and/or chemotherapy. Preferably, the target protein or peptide is a cytokine and/or chemokine. Even more preferably, the target protein or peptide is selected from the list comprising IL-15, IL-21, IFNα, CXCL9, CXCL10, IL-18, IL-27, or any combinations thereof. The skilled person will readily understand that the specific target protein or peptide that the heterologous polynucleotide encodes will be dependent on various factors, including but not limited to, cancer type, cancer severity and patient demographic.

The chemotherapy agent of the present invention may be any chemotherapy agent that prevents cancer cells from growing and dividing. Chemotherapy agents include, but are not limited to, alkylating agents, plant alkaloids, antitumour antibiotics, antimetabolites and/or topoisomerase inhibitors, or any combination thereof.

Alkylating agents work by attaching an alkyl group to DNA nucleotides, causing cross-linking of DNA strands, abnormal base pairing or DNA strand breaks which prevents DNA replication. Examples of alkylating agents include, but are not limited to, altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, and trabectedin.

Plant alkaloids work by interfering with DNA topoisomerases to inhibit DNA replication. Plant alkaloids may also bind to microtubule proteins during the metaphase stage of the cell cycle, thereby causing mitotic arrest and cell death. Examples of plant alkaloids include, but are not limited to, actinomycin D, doxorubicin, and mitomycin.

Antitumour antibiotics (also termed antineoplastic antibiotics) are commonly derived from Streptomyces bacteria and have a broad-spectrum anti-tumour effect against both solid and haematological tumours. Mechanisms of antitumour antibiotics include free-radical damage to DNA, topoisomerase II inhibition, DNA intercalation, alteration of ion transport, alteration of cell membrane fluidity and DNA binding. Examples of antitumour antibiotics include, but are not limited to bleomycin, dactinomycin, and anthracycline.

Antimetabolites work by mimicking nucleotide bases needed during DNA and RNA synthesis, thus interfering with mechanisms for cell proliferation, leading to cell death. Antimetabolites take effect primarily in rapidly dividing cells, such as tumour cells. Examples of antimetabolites include, but are not limited to, fludarabine, 5-fluorouracil, gemcitabine, cytarabine, pemetrexed.

Topoisomerase inhibitors inhibit cell proliferation by preventing DNA replication, stimulating DNA damage and inducing cell cycle arrest. There are two broad classes of topoisomerases: type I topoisomerases and type II topoisomerases. These enzymes plan an important role in cellular division and DNA organisation.

For example, topoisomerases mediate the cleavage of DNA strands to relax DNA supercoils and allow for DNA replication. Examples of topoisomerase inhibitors include, but are not limited to, irinotecan, topotecan, etoposide and teniposide.

Preferably, the chemotherapy agent is selected from the group comprising cyclophosphamide, gemcitabine, methotrexate, vinorelbine, docetaxel, bleomycin, vinblastine, dacarbazine, mustine, viscristine, procarbazine, prednisoline, etoposide, epirubicin, capecitabine, folinic acid, doxorubicin, carboplatin, cisplatin, daunorubicin, oxaliplatin, 5-fluorouracil, paclitaxel, mitomycin C, mitoxantrone, irinotecan, bleomycin, pemetrexed, trifluridine/tipiracil (TAS-102), anthracyclines, topoisomerase type II inhibitors, or any combination thereof. More preferably, the chemotherapy agent is selected from the group comprising cyclophosphamide, gemcitabine, cisplatin, carboplatin, methotrexate, vinblastine, doxorubicin, paclitaxel, oxaliplatin and mitomycin C, or any combination thereof. The skilled person will readily understand that the subject receiving the live attenuated Gram-negative and chemotherapy may also be receiving other therapies or medical interventions to enhance the efficacy of the treatment herein described.

The chemotherapy agent may be administered at a variety of doses. For example, the chemotherapy agent may be administered at a maximum effective dose. The maximum effective dose is the highest dose at which the chemotherapy agent is efficacious and has tolerable side effects. Accordingly, it is understood that the maximum effective dose will be specific to the chemotherapy agent of use and to the specific subject. Accordingly, the present invention enables the efficacy of the chemotherapy agent to be enhanced even when the maximum effective dose of the agent itself has been reached. Additionally, in some cases, the subject may benefit to such an extent from the improved efficacy of the chemotherapy agent that reduced cycles of the chemotherapy are required, thus reducing the chances of the subject experiencing adverse side effects due to the chemotherapy agent.

In other embodiments, the chemotherapy agent may be administered to the subject at a dose which is lower than the maximum effective dose, i.e., “a sub-maximal dose”, the efficacy of which is enhanced via the administration of the live attenuated Gram-negative bacteria, as described herein. Without being bound by theory, it is envisaged that administration of the chemotherapy agent at sub-maximal doses results in a particularly efficacious treatment when used in combination with the Gram-negative bacteria herein described due to such a dose damaging cancer cells opposed to killing them (Spiram et al., 2021, ScienceSignaling: 14:705). Having damaged cancer cells in the presence of a Salmonella-conditioned immune system is envisaged to enhance antigen presentation upon Salmonella-conditioning-induced increased uptake of damaged cancer cells by conditioned antigen presenting cells. Such changes are predicted to result in greater extent, magnitude and duration of specific adaptive anti-cancer immune responses. Accordingly, in these instances, chemotherapy induced toxicity is minimised, or at least reduced, from the start of the treatment regimen whilst still capable of producing the desired efficacy of the chosen chemotherapy.

The live attenuated Gram-negative bacteria and the chemotherapy of the present invention are administered to a subject in need of prophylactic or curative cancer treatment in a distinct temporal regimen. That is, the Gram-negative bacteria is administered in a first treatment phase and the chemotherapy is administered in a second treatment phase, i.e., the Gram-negative bacteria is administered to the patient or subject before the chemotherapy, and as such the immune system of the subject to be treated has the necessary time to be conditioned in response to the live attenuated Gram-negative bacteria, as described above. The invention herein described is therefore in stark contrast to the disclosure of Jia et al., 2007, where it was found that to avoid lethal toxicity, the bacteria must be administered 12 days after the administration of the chemotherapy. Therefore, the good safety profile of the treatment regimen in combination with the efficacious results herein described is a surprising finding of the present invention.

The present invention discloses the live attenuated Gram-negative bacteria being administered to a subject in a first treatment phase and a chemotherapy agent being administered to a subject in a second treatment phase. In a preferred embodiment, the first treatment phase is initiated at least one week prior to the second treatment phase. Even more preferably, the first treatment phase is initiated at least 10 days prior to the second treatment phase. For example, the first treatment phase may be initiated 7 days, 8 days, 9 days, 10 days, 11 days, 12 days, 13 days, 14 days, 15 days, 16 days, 17 days, 18 days, 19 days, 20 days, 21 days, 1 month, 2 months or 3 months prior to the start of the second treatment phase, i.e., the administration of the chemotherapy agent. Each of the treatment phases may comprise multiple administrations of either the Gram-negative bacteria or the chemotherapy agent. In a preferred embodiment, the live attenuated Gram-negative bacteria is administered prior to a chemotherapy cycle, with the possibility of repeating the administration of the Gram-negative bacteria prior to future additional chemotherapy cycles, as and when is required by the subject to be treated. It is further understood that the terms “first treatment phase” and “second treatment phase” refer to a single “cycle” of the preventative/curative treatment herein disclosed and that numerous cycles of said preventative/curative treatment are possible depending on the requirements of the subject in question.

The present invention provides for a live attenuated Gram-negative bacterium that can be used for the prevention and/or treatment of neoplastic disease, and/or secondary diseases associated with neoplastic disease, when used in combination with chemotherapy. In one embodiment, the neoplastic disease may be a solid cancer and/or a haematological malignancy. Neoplasia, tumours and cancers include benign, malignant, metastatic and non-metastatic types, and include any stage (I, II, III, IV or V) or grade (G1, G2, G3, etc.) of neoplasia, tumour, or cancer, or a neoplasia, tumour, cancer or metastasis that is progressing, worsening, stabilized or in remission.

It is envisaged that the live attenuated Gram-negative bacterium for use in the prevention or treatment of a neoplastic disease in a subject undergoing, or intended to undergo, chemotherapy with a chemotherapy agent will reduce metastasis of the primary tumour. In particular, prophylactic Salmonella treatment in combination with a chemotherapy agent reduces metastasis to the lungs.

Metastatic cancer is cancer which has spread from its primary site of origin to a secondary site in the body of a subject. The newly formed secondary pathological sites are termed metastases. The process of metastasis involves five stages of invasion, intravasation, circulation, extravasation, and colonisation. Tumour cells may undergo the transdifferentiation process of epithelial-mesenchymal transition, in which tumour cells may develop the ability to penetrate and invade nearby tissues. Such tumour cells which can pass through the basement membrane and extracellular matrix (at the primary site) can infiltrate into the lymphatic or vascular circulation (intravasation). Once in circulation, tumour cells may progress to the process of extravasation in which the vascular basement membrane and extracellular matrix are invaded at a secondary site, where the cells may attach to and colonise the secondary site. In addition to metastasising through the vasculature, tumour cells may directly invade nearby tissue surrounding the primary site.

Common sites of metastasis include the lungs, lymph nodes, liver and bone. Certain types of cancer may also be associated with some common sites of metastasis. For example, breast cancer may metastasise to the lung, liver, bones and/or brain. Bladder cancer may metastasise to the lung, liver and bones. Lung cancer may metastasise to the adrenal glands, bones, brain, liver, and/or the other lung.

Metastatic cancer may be classified as stage 3 cancer, in which the cancer has spread to a secondary site within the surrounding tissues or lymph nodes. Metastatic cancer may also be classified as stage 4 cancer, in which the cancer has spread to a secondary site within another organ which may be distant from the primary site. Metastatic cancers may be staged using the TNM system in which T describes the size of the tumour on a scale of 1 to 4; N describes the extent of spread to the lymph nodes on a scale of 0 to 3; and M describes the extent of metastasis on a scale of 0 to 1.

Cancers that may be treated according to the invention include, but are not limited to, cells or neoplasms of the bladder, blood, bone, bone marrow, brain, breast, colon, esophagus, gastrointestines, gum, head, kidney, liver, lung, nasopharynx, neck, ovary, prostate, skin, stomach, testis, tongue, or uterus. In addition, the cancer may specifically be of the following histological type, though it is not limited to the following: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumour, malignant; bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma with squamous metaplasia; thymoma, malignant; ovarian stromal tumour, malignant; thecoma, malignant; granulosa cell tumour, malignant; androblastoma, malignant; Sertoli cell carcinoma; Leydig cell tumour, malignant; lipid cell tumour, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumour; Mullerian mixed tumour; nephroblastoma; hepatoblastoma; carcinosarcoma; mesenchymoma, malignant; Brenner tumour, malignant; phyllodes tumour, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumour of bone; Ewing's sarcoma; odontogenic tumour, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumour; meningioma, malignant; neurofibrosarcoma; neurilemmoma, malignant; granular cell tumour, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Preferably, the solid cancer and/or the haematological malignancy may be a cancer selected from prostate cancer, liver cancer, renal cancer, lung cancer, breast cancer, colorectal cancer, bladder cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer or sarcoma.

In a preferred embodiment, the treatment regimen of the invention is to be administered to a patient with breast cancer with metastases, in particular where the metastases is in the lung.

Administration of the live attenuated Gram-negative bacterium in combination with a chemotherapy agent may reduce tumour burden, tumour growth, tumour progression and/or tumour metastases. The term “tumour burden” refers to the number of cancer cells, the size of a tumour or amount of tumour metastases in a subject. Reduced tumour burden is associated with improved treatment responses and survival outcomes. Reduced tumour burden may be indicated by, for example, a reduction in tumour volume, size or mass, a reduction in the rate of tumour growth, a reduction in the rate of tumour progression, a reduction in the rate of tumour metastasis, a reduction in the number of tumour metastasis, reduction in the number of tumours in a subject or overall reduction in the amount of cancer in a subject. Tumour burden may therefore be quantified, for example, by measuring tumour volume, tumour mass, tumour size (diameter, circumference, etc). Methods for quantifying tumour burden are known in the art, including but not limited to clinical imaging (MRI, PET-CT, etc.).

The amount of the live attenuated Gram-negative bacteria administered to the subject is sufficient to elicit a systemic immune response in the subject, so that the subject's immune system is effectively conditioned prior to receiving the chemotherapy agent. The immune response initiated by the administration of the Gram-negative bacteria may be of a therapeutic level in itself or be of a sub-therapeutic level wherein the subsequent administration of the chemotherapy agent is required for the desired response. The live attenuated Gram-negative bacteria may be administered at a dose of between 10⁴ and 10¹² CFU, where CFU is a colony-forming unit. For example, suitable doses may be between 10⁴ and 10⁵ CFU, 10⁴ and 10⁶ CFU, 10⁴ and 10⁷ CFU, 10⁴ and 10⁸ CFU, 10⁴ and 10⁹ CFU, 10⁴ and 10¹⁰ CFU, 10⁴ and 10¹¹ CFU, 10⁴ and 10¹² CFU, 10⁵ and 10⁶ CFU, 10⁵ and 10⁷ CFU, 10⁵ and 10⁸ CFU, 10⁵ and 10⁹ CFU, 10⁵ and 10¹⁰ CFU, 10⁵ and 10¹¹ CFU, 10⁵ and 10¹² CFU, 10⁶ and 10⁷ CFU, 10⁶ and 10⁸ CFU, 10⁶ and 10⁹ CFU, 10⁶ and 10¹⁰ CFU, 10⁶ and 10¹¹ CFU, 10⁶ and 10¹² CFU, 10⁷ and 10⁸ CFU, 10⁷ and 10⁹ CFU, 10⁷ and 10¹⁰ CFU, 10⁷ and 10¹¹ CFU, 10⁷ and 10¹² CFU, 10⁸ and 10⁹ CFU, 10⁸ and 10¹⁰ CFU, 10⁸ and 10¹¹ CFU, 10⁸ and 10¹² CFU, 10⁹ and 10¹⁰ CFU, 10⁹ and 10¹¹ CFU, 10⁹ and 10¹² CFU, 10¹⁰ and 10¹¹ CFU, 10¹⁰ and 10¹² CFU, or 10¹¹ and 10¹² CFU.

The live attenuated Gram-negative bacterium herein disclosed may, when in use, generate a systemic immune response in the subject. Preferably, the live attenuated Gram-negative bacterium generates a systemic immune response that results in an increase in the activation and/or maturation of myeloid cells. Examples of such myeloid cells include, but are not limited to, conventional dendritic cells, plasmacytoid dendritic cells, monocytes and/or macrophages. Accordingly, a systemic immune response in the context of the present invention may refer to long-term phenotypic changes in the circulating/systemic myeloid compartment which result in either an additive or synergistic effect when used in combination with a chemotherapy agent, thus improving patient outcomes. Other forms or readout of systemic immune response will be apparent to the skilled person and include Salmonella-specific antibody production and expansion of Salmonella-specific T cells. Measurements of such may be utilised to provide a measure of the efficacy of treatment.

In a second aspect of the present invention, there is a method of preventing or treating a neoplastic disease in a subject, wherein the method comprises administering to the subject, (i) a live attenuated Gram-negative bacterium in a first treatment phase and (ii) a chemotherapy agent in a second treatment phase, wherein said method results in an enhanced therapeutic efficacy relative to the administration of the bacterium or chemotherapy agent alone.

Therefore, the method of the present invention may be used to reduce or inhibit metastasis of a primary tumour or cancer to other sites, or the formation or establishment of metastatic tumours or cancers at other sites distal from the primary tumour or cancer thereby inhibiting or reducing tumour or cancer relapse or tumour or cancer progression. Accordingly, the present invention provides a detectable or measurable improvement in a condition of a given subject, such as alleviating or ameliorating one or more adverse (physical) symptoms or consequences associated with the presence of a cell proliferative or cellular hyperproliferative disorder, neoplasia, tumour or cancer, or metastasis, i.e., a therapeutic benefit or a beneficial effect.

The method of the present invention is therefore a combination therapy comprising administration of a live attenuated Gram-negative bacterium in a first treatment phase and a chemotherapy agent in a second treatment phase, with the potential to elicit potent and durable immune responses with enhanced therapeutic benefit. The additive or synergistic nature of the therapeutic combination herein disclosed may result in lower levels of chemotherapy being required, resulting in a reduction in adverse effects due to a more favourable toxicological profile.

A therapeutic benefit or beneficial effect is any objective or subjective, transient, temporary, or long-term improvement in the condition or pathology, or a reduction in onset, severity, duration or frequency of an adverse symptom associated with or caused by cell proliferation or a cellular hyperproliferative disorder such as a neoplasia, tumour or cancer, or metastasis. It may lead to improved survival. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumour or cancer, or metastasis.

A therapeutic benefit or improvement therefore may be, but is not limited to destruction of target proliferating cells (e.g., neoplasia, tumour or cancer, or metastasis) or ablation of one or more, most or all pathologies, adverse symptoms or complications associated with or caused by cell proliferation or the cellular hyperproliferative disorder such as a neoplasia, tumour or cancer, or metastasis. However, a therapeutic benefit or improvement need not be a cure or complete destruction of all target proliferating cells (e.g., neoplasia, tumour or cancer, or metastasis) or ablation of all pathologies, adverse symptoms or complications associated with or caused by cell proliferation or the cellular hyperproliferative disorder such as a neoplasia, tumour or cancer, or metastasis. For example, partial destruction of a tumour or cancer cell mass, or a stabilization of the tumour or cancer mass, size or cell numbers by inhibiting progression or worsening of the tumour or cancer, can reduce mortality and prolong lifespan even if only for a few days, weeks or months, even though a portion or the bulk of the tumour or cancer mass, size or cells remain.

Specific non-limiting examples of therapeutic benefit include a reduction in neoplasia, tumour or cancer, or metastasis volume (size or cell mass) or numbers of cells, inhibiting or preventing an increase in neoplasia, tumour or cancer volume (e.g., stabilizing), slowing or inhibiting neoplasia, tumour or cancer progression, worsening or metastasis, or inhibiting neoplasia, tumour or cancer proliferation, growth or metastasis.

An invention method may not take effect immediately. For example, treatment may be followed by an increase in the neoplasia, tumour or cancer cell numbers or mass, but over time eventual stabilization or reduction in tumour cell mass, size or numbers of cells in a given subject may subsequently occur.

Additional adverse symptoms and complications associated with neoplasia, tumour, cancer and metastasis that can be inhibited, reduced, decreased, delayed or prevented include, for example, nausea, lack of appetite, lethargy, pain and discomfort. Thus, a partial or complete decrease or reduction in the severity, duration or frequency of an adverse symptom or complication associated with or caused by a cellular hyperproliferative disorder, an improvement in the subjects quality of life and/or well-being, such as increased energy, appetite, psychological well-being, are all particular non-limiting examples of therapeutic benefit.

A therapeutic benefit or improvement therefore can also include a subjective improvement in the quality of life of a treated subject. In an additional embodiment, a method prolongs or extends lifespan (survival) of the subject. In a further embodiment, a method improves the quality of life of the subject.

It is envisaged that the present invention may be particularly suited to individuals who have been refractory to previous treatment with a chemotherapy. By “refractory”, we intend a reference to any neoplastic disease that does not respond to treatment, for example, a chemotherapy agent. It is also envisaged that the present invention will be particularly suited to individuals who have previously been low responders, moderate responders or high responders to previous chemotherapy treatments. Determination of whether a subject is a responder or a non-responder may be based on their previous response to chemotherapy treatment, or via biomarker analysis of a biological sample to identify markers which are indicative of a subject responding, or not responding, to a certain treatment.

The inventors of the present invention have surprisingly found that administering live attenuated Gram-negative bacteria to a subject prior to the administration of a chemotherapy agent results in the enhanced efficacy of said chemotherapy. The invention is further described with reference to the following non-limiting examples:

### EXAMPLES

### Example 1—Conditioning a Subject's Immune System with Gram-Negative Bacteria Enables Enhanced Response Rates to Chemotherapy

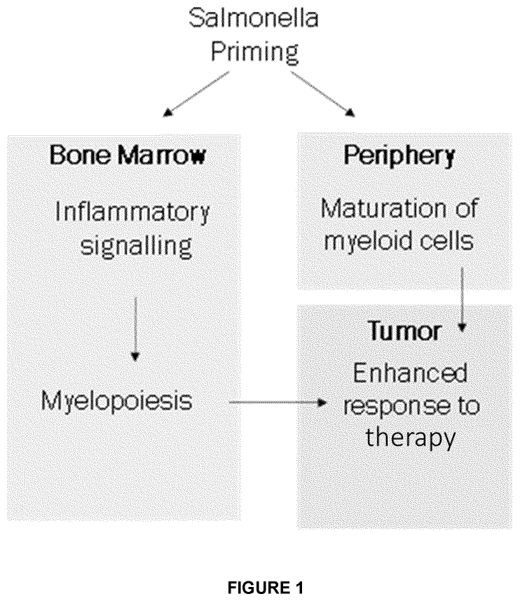

The present invention provides a method in which Gram-negative bacteria can be used to effectively and systemically condition the immune system of a subject such that the efficacy of a subsequently administered chemotherapy agent is enhanced. Without being bound by theory, a possible mechanism of action demonstrating how such an effect occurs is provided for in FIG. 1.

### Example 2—Induction of Long-Term Phenotypic Changes in Systemic Myeloid Cells

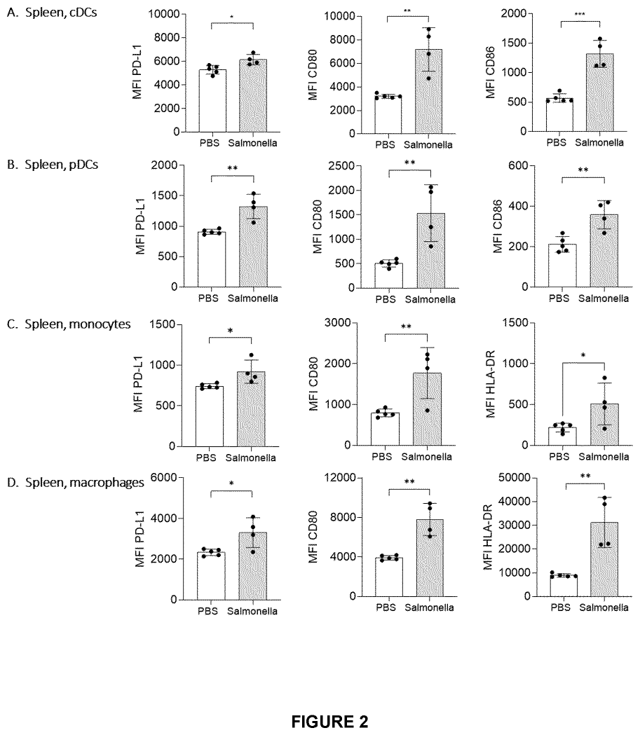

Adult, female BALB/c mice were treated with 1×10⁹ CFU Salmonella enterica serovar Typhimurium strain MD58 (ΔaroC) orally. 21 days later spleens were harvested, single cell suspensions generated and flow cytometry staining performed. Median fluorescence intensity of markers CD80, CD86 and PD-L1 on viable, CD11c^{high}, HLA-DR⁺, CD11b^{+/-}, PDCA-1⁻ conventional dendritic cells and viable, CD11c^{-/low}, PDCA1⁺, HLA-DR^{-/Int}, CD11b⁻ plasmacytoid dendritic cells was measured (see FIGS. 2A and 2B). Median fluorescence intensity of markers PD-L1, CD80 and HLA-DR on viable, CD11c⁻, CD11b⁺, Ly6C⁺, F4/80⁻ monocytes and viable, CD11c⁻, CD11b⁺, Ly6C⁻, F4/80⁺ macrophages was also measured (see FIGS. 2C and 2D).

As can be seen FIG. 2, various cell markers of myeloid cells, for example, conventional dendritic cells, plasmacytoid dendritic cells, monocytes and macrophages displayed a significant increase at 21 days following treatment with Salmonella, suggesting that the effect on activation of immune cells following treatment with Salmonella is sustainable over a significant period of time.

### Example 3—Time-Course of Salmonella-Induced Phenotypic Changes

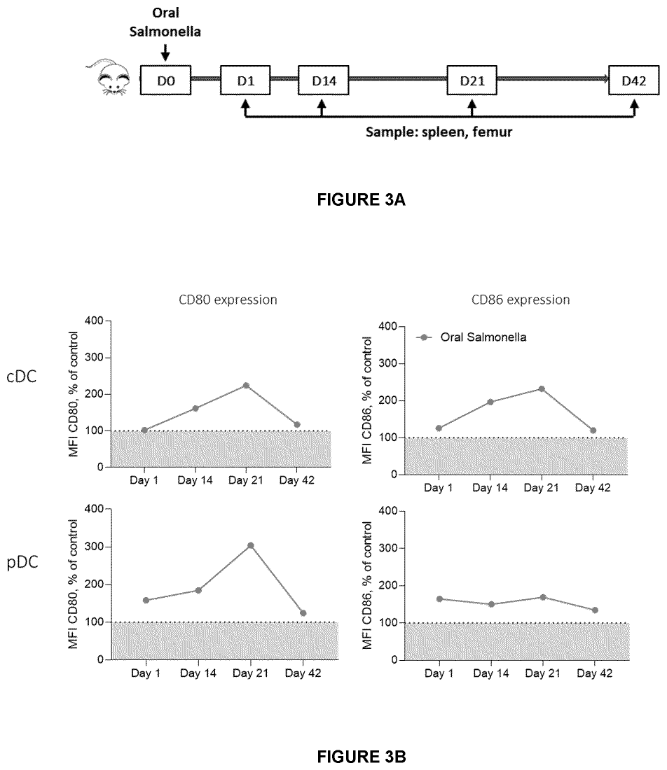

To explore the kinetics of activation/maturation phenotypic changes on myeloid cells observed in FIG. 2, we performed a time-course study. Adult, female BALB/c mice were treated with 1×10⁹ CFU Salmonella enterica serovar Typhimurium strain MD58 (ΔaroC) orally. 1, 14, 21 or 42 days later spleens and femurs were harvested, single cell suspensions generated and flow cytometry staining performed (see experiment schematic of FIG. 3A).

Activation markers on systemic conventional (CDC) and plasmacytoid (pDC) dendritic cells were shown to be up-regulated following oral administration of Salmonella, peaking at 3 weeks post-administration and returning close to baseline levels by 6 weeks post-administration (see FIG. 3B). Activation markers on systemic monocytes and macrophages were also shown to be up-regulated following oral administration of Salmonella, peaking at 3 weeks post-administration and returning close to baseline levels by 6 weeks post-administration (see FIG. 3C).

### Example 4—Oral Administration of Salmonella Enhances Myelopoiesis

Adult, female BALB/c mice were treated with 1×10⁹ CFU Salmonella enterica serovar Typhimurium strain MD58 (ΔaroC) orally. 1, 14, 21 or 42 days later flow cytometry staining of isolated bone marrow cells was performed.

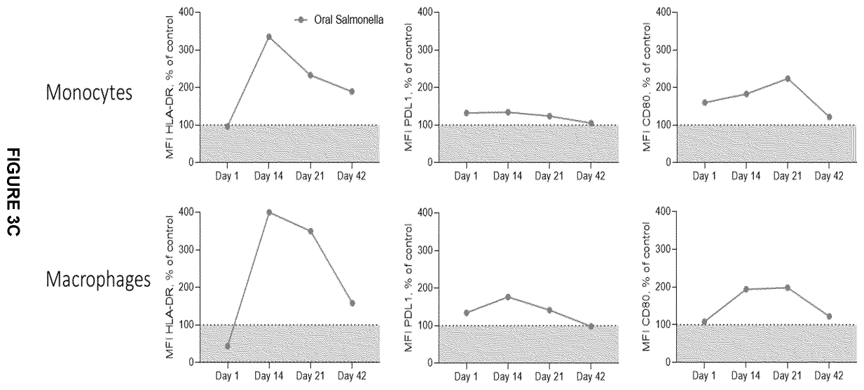

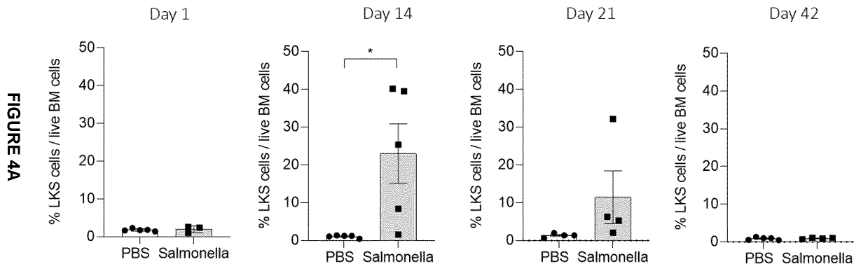

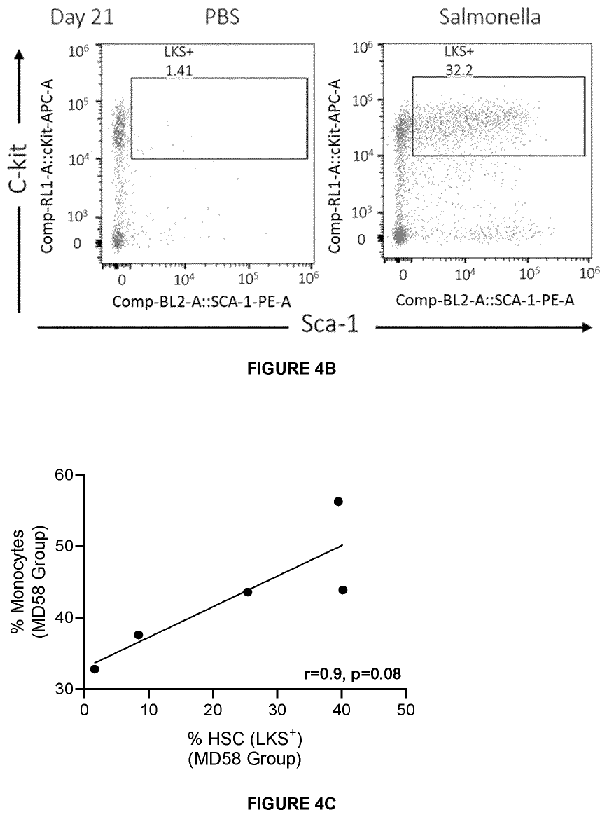

The number of hematopoietic stem cells/multipotent progenitors in the bone marrow is shown to increase significantly 2 weeks after oral administration (see FIG. 4A). Representative flow cytometry plots also demonstrate an increase in viable LKS cells in the bone marrow of animals treated orally with Salmonella (see FIG. 4B). Additionally, the % of viable LKS cells of total bone marrow cells at day 14 after Salmonella treatment was correlated with % monocytes (viable, CD11c⁻, CD11b⁺, Ly6C⁺, F4/80⁻ cells) at the same timepoint using Spearman rank correlation (see FIG. 4C).

Accordingly, the data herein supports the hypothesis that administration of Salmonella conditions the immune system, and in particular the myeloid arm of the immune system. Furthermore, these data demonstrate long-term effects of Salmonella conditioning on the immune system, as demonstrated by the 3-6 weeks duration of conditioning changes induced by Salmonella. These changes are likely systemically/centrally mediated, as suggested by the positive correlation between the number of bone marrow hematopoietic progenitor cells and splenic monocytes.

### Example 5—Oral Administration of Salmonella Induces a Hyperresponsive State in Systemic Dendritic Cells Lasting at Least 14 Days

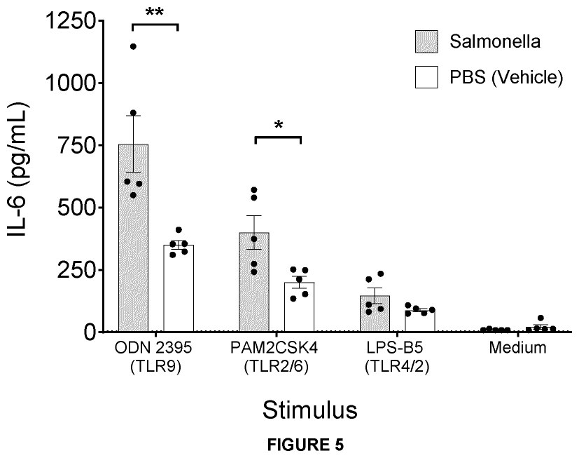

Adult, female BALB/c mice were treated with 1×10⁹ CFU Salmonella enterica serovar Typhimurium strain MD58 (ΔaroC) orally. 14 days later spleens were harvested, single cell suspensions generated, CD11c expressing cells were enriched by magnetic separation and 1×10⁵ cells/well were incubated with the indicated stimuli (TLR9, TLR2/6, or TLR4/2 agonists or a control) for 24 hrs. IL-6 in the supernatant was measured by LegendPlex assay (see FIG. 5).

As is evident from FIG. 5, CD11c expressing cells from animals that had been treated with a Gram-negative bacteria, for example, Salmonella, demonstrated enhanced IL-6 secretion (an indicator of the level of immune responsiveness of a cell) in response to a variety of stimuli when compared to the vehicle control group. This further supports that the administration of Gram-negative bacteria of the present invention results in conditioning of immune cells, rendering them more responsive to subsequent stimuli, for example, DAMPS induced by a chemotherapy agent.

As demonstrated in Examples 1 to 5, the experimental data herein disclosed shows that treatment with a live attenuated Gram-negative bacterium, for example, Salmonella, results in an unexpected long-term activation of multiple immune cell types (e.g. dendritic cells, monocytes and macrophages), as well as an increase in hematopoietic stem cells/multipotent progenitors in the bone marrow. Without being bound by theory, it is believed that the systemic response induced by administration of such a live attenuated Gram-negative bacterium is able to condition the immune system of a subject or patient such that when administered in combination with a chemotherapy agent, the anti-tumour activity of said chemotherapy is enhanced. It is believed that the systemic modifications observed from administering the live attenuated Gram-negative bacteria, i.e., the activation of multiple immune cell types and effect on myelopoiesis (as demonstrated herein), in combination with the intestinal uptake of said live attenuated Gram-negative bacteria is responsible for the enhanced anti-tumour effect. The skilled person will readily understand that the broad systemic effects induced by the live attenuated Gram-negative bacterium may condition the immune system of a patient/subject in such a way that the advantageous effects are observed regardless of the chemotherapy agent to be used. Additionally, the use of Gram-negative bacteria to condition the immune system of a subject/patient may be particular advantageous for those patients/subjects who have been shown to be resistant to chemotherapies when used in isolation.

### Example 6—Conditioning Primary Human Monocytes In Vitro with a Gram-Negative Bacteria Results in Hyperresponsiveness to Subsequent Challenge with PAMPs and DAMPS

Primary human monocytes were conditioned in vitro with Salmonella for 30 minutes prior to being washed with an antibiotic to remove all Salmonella and subsequently rested for 6 days in the presence of M-CSF. Following the 6 day rest period, the human monocytes were stimulated with PAMPs, DAMPs or a media only control, and the level of TNFα release measured via ELISA.

The inventors of the present invention have surprisingly found that cells conditioned with the Gram-negative bacteria herein disclosed show hyperresponsiveness to both PAMPs and DAMPs (FIG. 6), as reflected in the level of release of TNFα. This is in contrast to the established conditioning agent β-glucan that only induces hyperresponsiveness in response to challenge with PAMPs and shows no difference in the level of release of TNFα to cells treated with media (negative control). On the other hand, cells conditioned with the Gram-negative bacteria herein disclosed and subsequently challenged with HMGB1 (a known DAMP) showed approximately a 5-fold increase in TNFα release compared to the media only control.

### Example 6—Salmonella Treatment in Combination with Cyclophosphamide, Gemcitabine or Doxorubicin

### Methods

### Bacterial Cell Preparation

Dilutions of the attenuated Salmonella Typhimurium strain (MD58) were made in PBS as required to achieve 1×10⁹ CFU/100 μl for oral gavage.

### Orthotopic 4T1 Breast Tumour Model

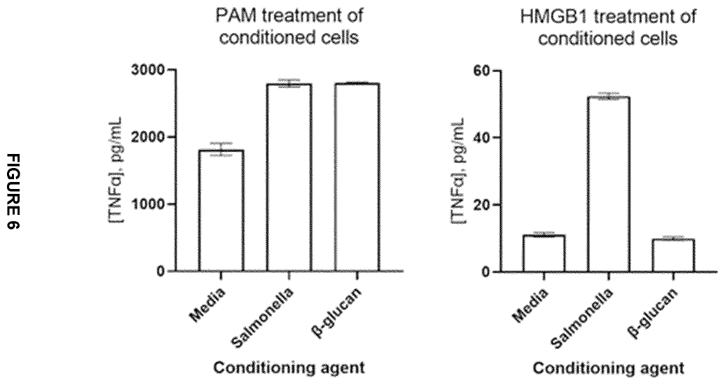

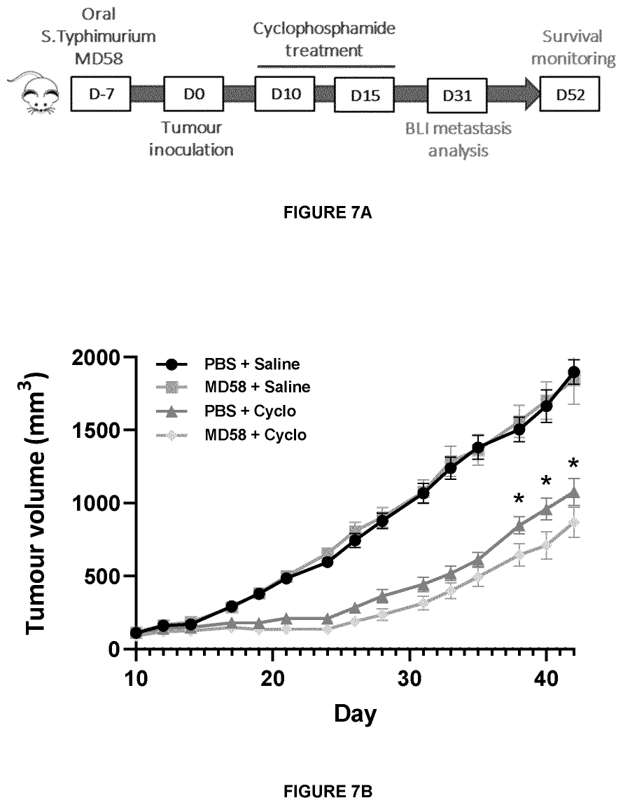

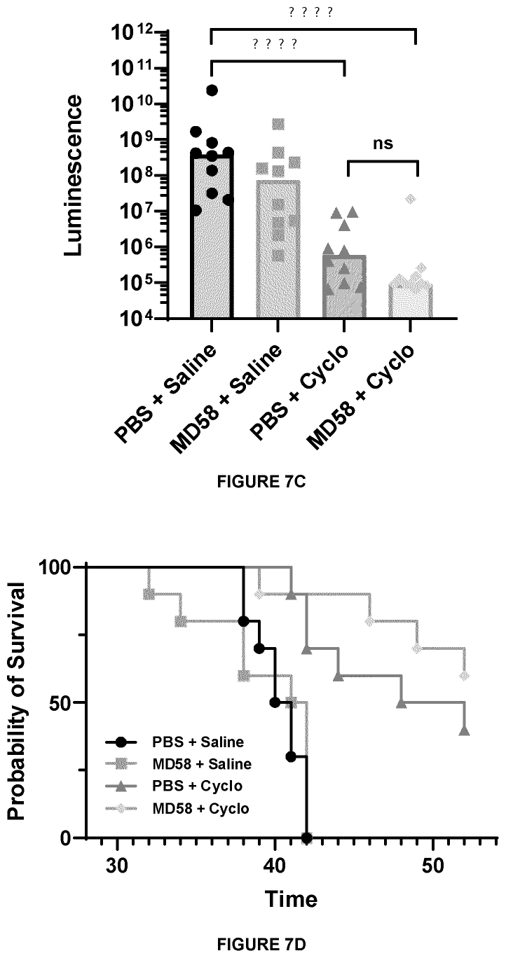

6-7-week-old female BALB/c mice were pre-treated orally with 1×10⁹ CFU Salmonella MD58 or PBS control. On day 0 mice were inoculated in mammary fat pad #4 with 5×10⁵ 4T1-Luc2-1A4 tumour cells. On day 10 and 15 post tumour challenge mice received 100 mg/kg cyclophosphamide intraperitoneally (IP) (FIG. 7A). Primary tumour volumes were measured 3 times per week, lung tumour burden was measured by bioluminescent imaging (BLI) and survival was monitored until 52 days after tumour inoculation

### Experimental 4T1 Lung Metastasis Model

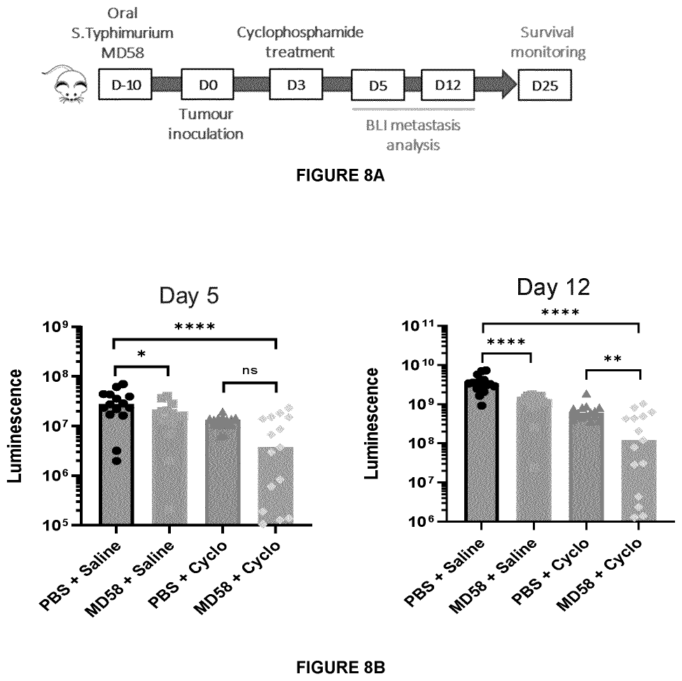

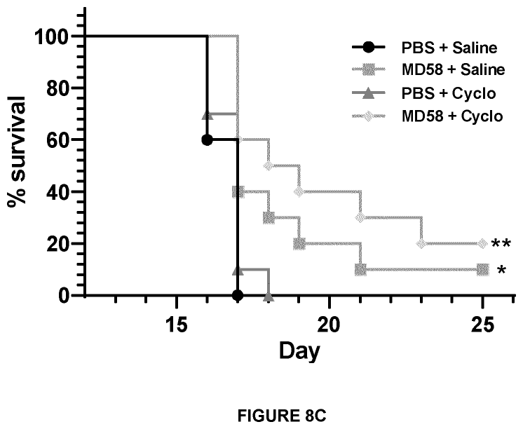

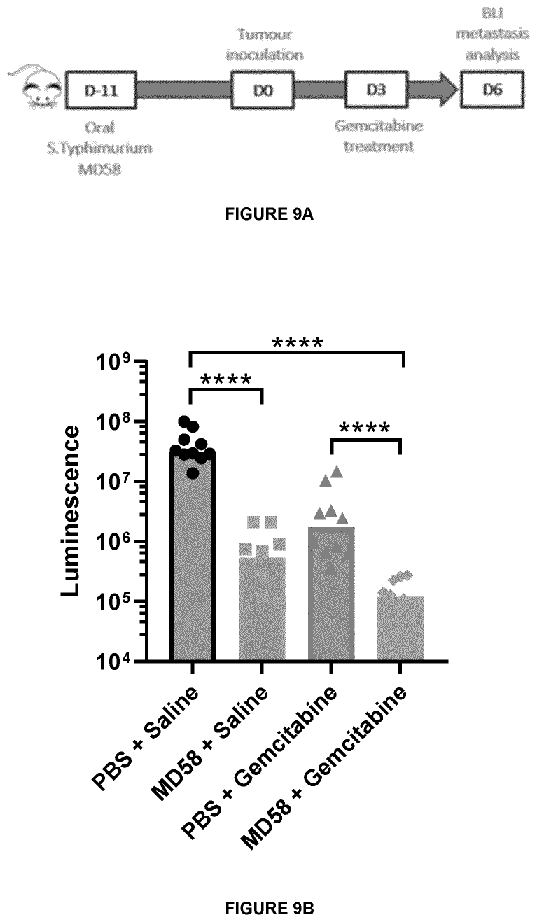

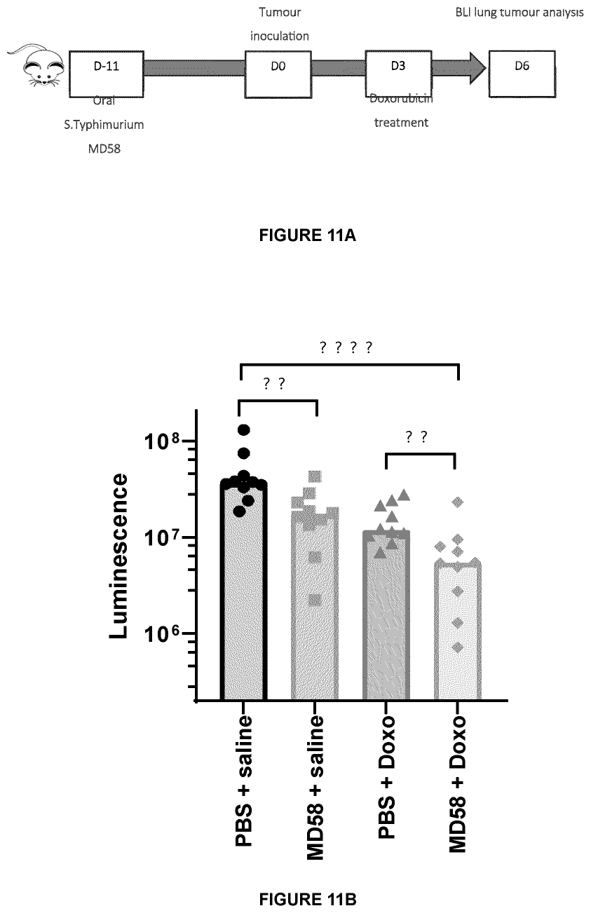

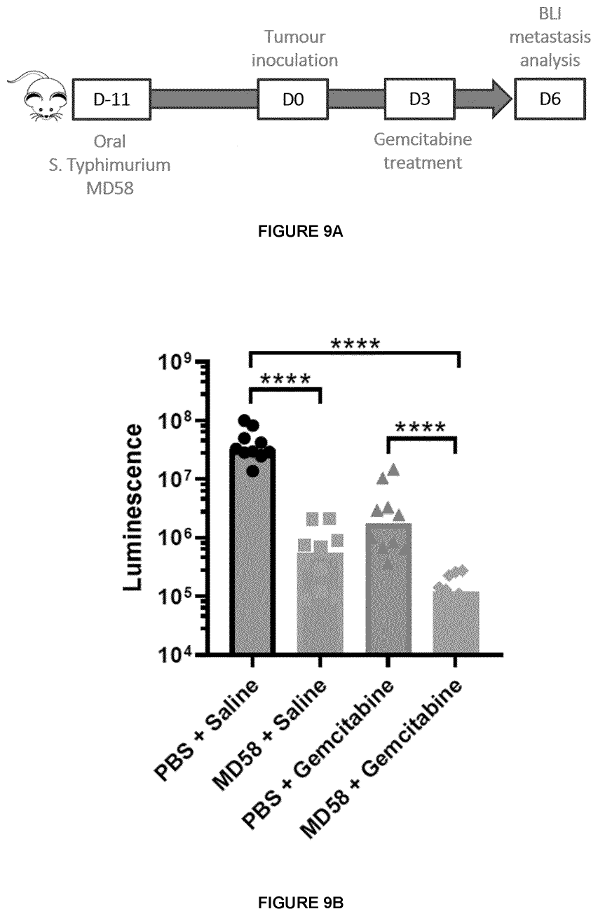

6-7-week-old female BALB/c mice were pre-treated orally with 1×10⁹ CFU Salmonella MD58 or PBS control. On day 0 mice were inoculated intravenously with 1×10⁵ 4T1-Luc2-1A4 tumour cells. On day 3 post tumour challenge mice received either 40 mg/kg cyclophosphamide (FIG. 8A) or 60 mg/kg gemcitabine (FIG. 9A) intraperitoneally (IP) or 10 mg/kg doxorubicin intravenously (IV) (FIG. 11A). Lung tumour growth was monitored by bioluminescent imaging (BLI) and survival was monitored.

### LL/2 Lung Tumour Model

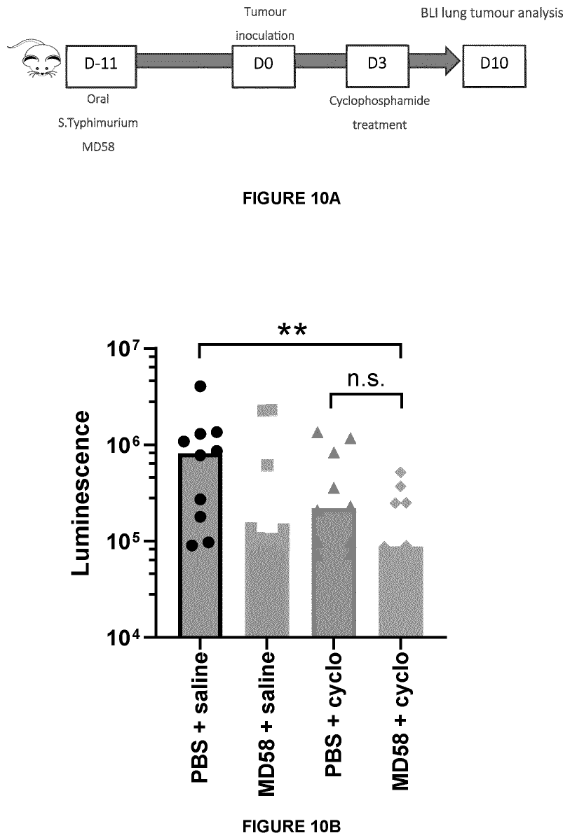

6-7-week-old female albino C57BL/6 mice were pre-treated orally with 1×10⁹ CFU Salmonella MD58 or PBS control. On day 0 mice were inoculated intravenously with 1×10⁵ LL/2-Luc-M38 tumour cells. On day 3 post tumour challenge mice received 40 mg/kg cyclophosphamide intraperitoneally (IP) (FIG. 10A). Lung tumour growth was monitored by bioluminescent imaging (BLI).

### In Vivo Bioluminescence Imaging

Lung metastasis in all models were monitored by bioluminescence imaging (BLI). 10 minutes after intraperitoneal (IP) injection of D-luciferin (Promega, E1605, 3 mg/20 g of body weight) mice were imaged in the IVIS Spectrum (Perkin Elmer, Waltham, MA) using a single thoracic fixed-area ROI for each individual animal. Image analysis was done using Living Image 4.7.1 (Perkin Elmer, Waltham, MA). Binning and exposures times were adjusted to obtain at least several hundred counts per image and to avoid saturation of the CCD chip.

### Results

Oral administration of Salmonella Typhimurium, followed by systemic administration of cyclophosphamide in a syngeneic orthotopic 4T1 murine breast cancer model reduced primary orthotopic breast tumour burden, lung metastases (n.s.) and improves survival (n.s.) compared to cyclophosphamide monotherapy (FIGS. 7A-D).

Oral systemic administration of Salmonella Typhimurium, followed by intravenous administration of 4T1 murine breast cancer cell, and then followed by systemic cyclophosphamide reduced experimental lung metastasis in 4T1 tumour-bearing mice and reduced lung tumour burden at day 5 and 12 post tumour inoculation (FIGS. 8A-C).

Oral systemic administration of Salmonella Typhimurium, followed by intravenous administration of 4T1 murine breast cancer cell, and then followed by intraperitoneal gemcitabine reduced experimental 4T1 metastasis to the lungs (FIGS. 9A-B).

Oral systemic administration of Salmonella Typhimurium, followed by intravenous administration of LL/2-Luc-M38 murine lung cancer cell, and then followed by systemic cyclophosphamide reduced LL/2 lung tumour growth (FIGS. 10A-B). Oral systemic administration of Salmonella Typhimurium, followed by intravenous administration of 4T1 murine breast cancer cell, and then followed by intravenous doxorubicin reduced experimental 4T1 metastasis to the lungs (FIGS. 11A-B).

Accordingly, the inventors of the present invention have shown that administration of a Gram-negative bacteria in a first treatment phase, followed by administration of a chemotherapy agent in a second treatment phase, enables a more efficacious therapy than if either treatment was used in isolation. As such, the present invention provides a method by which cancer patient outcomes can be improved.

1-20. (canceled)

21. A method of preventing or treating a neoplastic disease in a subject, wherein the method comprises administering to the subject,
(i) a live attenuated Gram-negative bacterium in a first treatment phase, wherein the live attenuated Gram-negative bacterium does not further comprise a heterologous polynucleotide encoding a polypeptide, wherein the polypeptide is a therapeutic molecule, and
(ii) a chemotherapy agent in a second treatment phase,
wherein said method results in an enhanced therapeutic efficacy relative to the administration of the bacterium or chemotherapy agent alone.

22. The method of claim 21, wherein the live attenuated Gram-negative bacterium is formulated for oral administration.

23. The method of claim 21, wherein the live attenuated bacterium is a Salmonella spp.

24. The method of claim 23, wherein the live attenuated bacterium is Salmonella enterica.

25. The method of claim 24, wherein the live attenuated bacterium is Salmonella enterica serovar Typhi and/or Salmonella enterica serovar Typhimurium.

26. The method of claim 21, wherein the bacterium is a genetically modified non-natural bacterium.

27. The method of claim 21, wherein the genetically modified non-natural bacterium comprises an attenuating mutation in a Salmonella Pathogenicity Island 2 (SPI-2) gene and an attenuating mutation in a second gene.

28. The method of claim 27, wherein the SPI-2 gene is a ssaV gene and the second gene is an aro gene.

29. The method of 21, wherein the chemotherapy agent is selected from the group comprising methotrexate, vinorelbine, docetaxel, bleomycin, vinblastine, dacarbazine, mustine, viscristine, procarbazine, prednisoline, etoposide, epirubicin, capecitabine, folinic acid, doxorubicin, carboplatin, cisplatin, cyclophosphamide, daunorubicin, oxaliplatin, 5-fluorouracil, gemcitabine, paclitaxel, mitomycin C, mitoxantrone, irinotecan, bleomycin, pemetrexed, trifluridine/tipiracil (TAS-102), anthracyclines, topoisomerase 2 inhibitors, and any combination thereof.

30. The method of claim 29, wherein the chemotherapy agent is selected from the group comprising cisplatin, gemcitabine, carboplatin, methotrexate, vinblastine, doxorubicin, paclitaxel, oxaliplatin, mitomycin C, and any combination thereof.

31. The method of claim 21, wherein the first treatment phase is initiated to allow sufficient time to generate a systemic immune response in the subject prior to the second treatment phase.

32. The method of claim 31, wherein the first treatment phase is initiated at least one week prior to the second treatment phase.

33. The method of claim 32, wherein the first treatment phase is initiated at least ten days prior to the second treatment phase.

34. The method of claim 30, wherein the first treatment phase is initiated 7 days, 8 days, 9 days, 10 days, 11 days, 12 days, 13 days, 14 days, 15 days, 16 Page 5 of 6 days, 17 days, 18 days, 19 days, 20 days, 21 days, 1 month, 2 months or 3 months prior to the second treatment phase.

35. The method of claim 21, wherein the neoplastic disease is associated with a solid tumour and/or haematological malignancy.

36. The method of claim 35, wherein the neoplastic disease is associated with a cancer selected from prostate cancer, liver cancer, renal cancer, lung cancer, breast cancer, colorectal cancer, bladder cancer, pancreatic cancer, brain cancer, hepatocellular cancer, lymphoma, leukaemia, gastric cancer, cervical cancer, ovarian cancer, thyroid cancer, melanoma, carcinoma, head and neck cancer, skin cancer or sarcoma.

37. The method of claim 21, wherein the subject has breast cancer with metastases in the lung.
